(19) United States
(12) Patent Application Publication
(10) Pub. No.: US 2026/0053375 A1
(43) Pub. Date: Feb. 26, 2026

(54) SYSTEM AND METHOD FOR CAMERA-BASED REMOTE BLOOD PRESSURE MONITORING

(71) Applicant: The Hong Kong University of Science and Technology, Hong Kong (CN)

(72) Inventors: Kwan Long WONG, Hong Kong (CN); Jing Wei CHIN, Hong Kong (CN); Richard Hau Yue SO, Hong Kong (CN); Chun Hong CHENG, Hong Kong (CN); Tsz Tai CHAN, Hong Kong (CN)

(21) Appl. No.: 19/260,311

(22) Filed: Jul. 4, 2025

Related U.S. Application Data
(60) Provisional application No. 63/685,266, filed on Aug. 21, 2024.

(51) Int. Cl. A61B 5/021; A61B 5/00; G16H 15/00

(52) U.S. Cl. CPC A61B 5/021; A61B 5/0002; A61B 5/0077; A61B 5/7267; G16H 15/00

(57) ABSTRACT

In the present invention, to extract the subject's SBP and DBP estimation, the system first starts capturing a color video of the subject. Then, computer vision techniques are applied to the video frames to locate regions of interest on the face, and these regions are tracked continuously for a while. Next, the images with located region of interest are fed into a pipeline with image processing, signal processing, and machine learning algorithms to create a signal based on the rPPG signal. The rPPG signal, along with its derivatives of different orders and an estimated HR from the rPPG signal are all fed into a machine learning model to estimate/predict the SBP and DBP of the subject.

### CROSS-REFERENCE TO RELEVANT APPLICATIONS

The present application claims priority from a U.S. provisional patent application Ser. No. 63/685,266 filed Aug. 21, 2024, and the disclosure of which is incorporated by reference in its entirety.

### TECHNICAL FIELD

This invention is related to the field of computer vision, which is used to extract physiological-related information from images and then estimate the systolic and diastolic blood pressure of a subject with the use of a machine learning model.

### BACKGROUND

Blood pressure (BP) is a crucial physiological factor for identifying any potential cardiovascular disease in a subject. It is usually assessed by systolic blood pressure (SBP) and diastolic blood pressure (DBP), where the former represents the pressure in arteries when the heart beats, and the latter represents the pressure in arteries when the heart is resting between beats.

Cuff-based methods based on oscillometry and sphygmomanometers have been generally used for measuring BP over the past decades. However, they are not suitable for long-term monitoring as they may cause discomfort and irritation to people with sensitive skin. To overcome these limitations, cuffless methods have been developed for continuous BP monitoring, especially utilizing signals produced by photoplethysmography (PPG-signals) and machine learning methods. PPG signals are frequently used to assess other vital signs as well, including heart rate (HR), respiratory rate, and blood oxygen saturation. They involve employing a light emitter and sensor to gauge the fluctuations in blood vessel volume beneath the skin. When tissue is exposed to light, the photodetector records slight changes in light intensity caused by blood flow.

However, these approaches remain contact-based and do not provide a pain-free solution. Consequently, contactless methods for BP measurement have become highly desirable. There is a need for a contactless BP measurement system that can enhance comfort while achieving accuracy for health monitoring.

### SUMMARY OF INVENTION

It is an objective of the present invention to provide a system and a method to solve the aforementioned technical problems for extraction of physiological-related information from images.

Briefly, to extract the subject's systolic blood pressure (SBP) and diastolic blood pressure (DBP), the system first starts capturing a color video of the subject. Then, computer vision techniques are applied to the video frames to locate regions of interest on the face, and these regions are tracked continuously for a while. Next, the images with located region of interest are fed into a pipeline with image processing, signal processing, and machine learning algorithms to create a signal based on the remote photoplethysmography (rPPG signal). The rPPG signal, along with its derivatives of different orders and an estimated HR from the rPPG signal are all fed into a machine learning model to estimate/predict the SBP and DBP of the subject.

In accordance with a first aspect of the present invention, a system for camera-based remote blood pressure monitoring is provided. The system includes a camera, a color image and signal processing system, a rPPG-signal extraction system, and a blood pressure estimator. The camera is configured to capture color image frames of a subject. The color image and signal processing system is configured to extract physiological signals according to the color image frames of the subject by: executing light intensity analysis for the color image frames of the subject; identifying a location of face and facial landmarks of the subject; tracking regions of interest (ROIs) based on the approximated facial landmarks; selecting one of the regions of interest; and extracting the physiological signals from the selected region of interest. The rPPG-signal extraction system is configured to use the extracted physiological signals to generate a rPPG signal embedding the subject's cardiovascular activity information by: forming ID (one-dimension) signals from the extracted physiological signals; applying machine learning or image processing algorithms to obtain a rPPG signal using the 1D signals; applying bandpass filtering on the extracted rPPG signal; and computing derivatives of different orders of the rPPG signal to generate a derivative information. The blood pressure estimator receives the rPPG signal and the derivative information and is configured to output an estimated SBP and DBP of the subject based on the rPPG signal and the derivative information using a well-trained machine learning model. The report output module is configured to receive the estimated SBP and DBP of the subject and to provide a readable report regarding the subject's SBP and DBP estimation values.

In accordance with a second aspect of the present invention, a method for camera-based remote blood pressure monitoring is provided. The method includes steps as follows: capturing color image frames of a subject using a camera; extracting physiological signals according to the color image frames of the subject using a color image and signal processing system by steps of: (1) executing light intensity analysis for the color image frames of the subject; (2) identifying a location of face and facial landmarks of the subject; (3) tracking ROIs based on the approximated facial landmarks; (4) selecting one of the regions of interest; and (5) extracting the physiological signals from the selected region of interest. The method further includes using the extracted physiological signals to generate a rPPG signal embedding the subject's cardiovascular activity information, using a physiological activity image processing system, by steps of: (1) forming ID (one-dimension) signals from the extracted physiological signals; (2) applying machine learning or image processing algorithms to obtain a rPPG signal using the ID signals; (3) applying bandpass filtering on the extracted rPPG signal; and (4) computing derivatives of different orders of the rPPG signal to generate a derivative information. The method further includes: receiving the rPPG signal and the derivative information by a blood pressure estimator; outputting, by the blood pressure estimator, an estimated SBP and DBP of the subject based on the rPPG signal and the derivative information using a well-trained machine learning model; and receiving, by a report output module, the estimated SBP and DBP of the subject and provide a readable report regarding the subject's SBP and DBP estimation values.

By the configuration, the technical effect provided by the system with machine learning models is the ability to perform contactless blood pressure monitoring using a camera. This method provides a pain-free and non-invasive solution for continuous blood pressure monitoring, overcoming the limitations of traditional cuff-based methods. By leveraging computer vision and machine learning, the system can accurately estimate blood pressure from video frames, making it suitable for long-term monitoring and improving user comfort.

### BRIEF DESCRIPTION OF DRAWINGS

Embodiments of the invention are described in more details hereinafter with reference to the drawings, in which:

FIG. 1 illustrates a schematic diagram of an architecture of a camera-based remote blood pressure monitoring system according to some embodiments of the present invention;

FIG. 2 shows a schematic diagram of a process of estimating the SBP and DBP according to some embodiments of the present invention;

FIG. 3 shows a schematic diagram of a process for converting raw color frames into physiological signals using a color image and signal processing system according to some embodiments of the present invention;

FIG. 4 shows a schematic diagram of a process for generating a rPPG signal from extracted physiological signals using a rPPG-signal extraction system of the subject according to some embodiments of the present invention;

FIG. 5 illustrates a process of forming a rPPG signal from a captured video of a subject according to some embodiments of the present invention;

FIG. 6 illustrates a process of training a machine learning model of a blood pressure estimator using a captured video of a subject according to some embodiments of the present invention; and

FIG. 7 illustrates a schematic diagram of an architecture of a camera-based remote blood pressure monitoring system according to some embodiments of the present invention.

### DETAILED DESCRIPTION OF THE INVENTION

In the following description, systems and methods for camera-based remote blood pressure monitoring and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

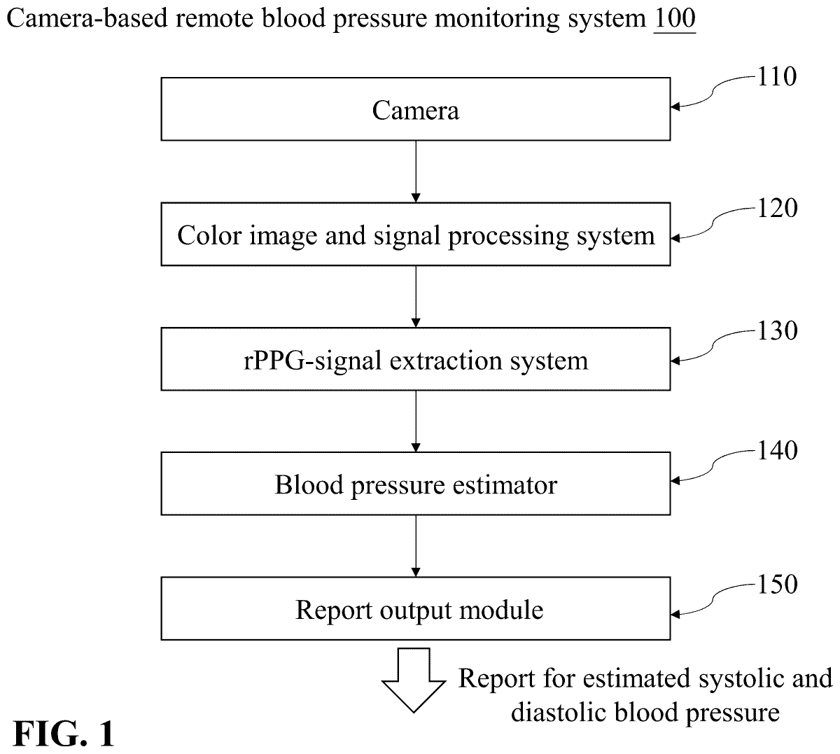

FIG. 1 illustrates a schematic diagram of an architecture of a camera-based remote blood pressure monitoring system 100 according to some embodiments of the present invention. The system 100 includes a camera 110, a color image and signal processing system 120, a remote photoplethysmography-signal (rPPG-signal) extraction system 130, a blood pressure estimator 140, and a report output module 150.

The camera 110 is configured to capture a video with color image frames of a subject to be analyzed. Herein, the term “subject” refers to a human undergoing blood pressure monitoring. The camera 110 serves as a primary input device for the system 100, providing raw visual data required for subsequent processing steps. In some embodiment, the camera 110 may record one or more color videos with color image frames for the subject.

The color image and signal processing system 120 communicates with the camera 110, in which the color image frames captured by the camera 110 serve as input to the color image and signal processing system 120. The color image and signal processing system 120 is configured to extract physiological signals according to the color image frames of the subject. Specifically, during the processing on the color image frames, the color image and signal processing system 120 can perform several key functions. The color image and signal processing system 120 is configured to: execute light intensity analysis for the color image frames of the subject; identify the location of the face and facial landmarks of the subject; track the regions of interest (ROIs) based on the approximated facial landmarks; select the best region of interest based on certain metrics; extract physiological signals from the selected region of interest.

The rPPG-signal extraction system 130 communicates with the color image and signal processing system 120, in which the physiological signals are transferred to the rPPG-signal extraction system 130 from the color image and signal processing system 120 after extraction. The rPPG-signal extraction system 130 is configured to use the extracted physiological signals to generate a rPPG signal embedding the subject's cardiovascular activity information. Specifically, during the generation of the rPPG signal, the rPPG-signal extraction system 130 can perform several key functions. The rPPG-signal extraction system 130 is configured to: form 1D (one-dimension) signals from the extracted physiological signals; apply machine learning or image processing algorithms to obtain a rPPG signal using the 1D signals; apply bandpass filtering on the extracted rPPG signal; compute derivatives of different orders of the rPPG signal to generate a derivative information.

The blood pressure estimator 140 communicates with the rPPG-signal extraction system 130 for receiving the rPPG signal and the derivative information. The rPPG signal, along with its derivatives of different orders (i.e., the derivative information), and an estimated heart rate (HR) computed from the rPPG signal, are all fed into the blood pressure estimator 140 for inferencing, which outputs an estimated systolic blood pressure (SBP) and diastolic blood pressure (DBP) of the subject. Specifically, the blood pressure estimator 140 includes a machine learning model set up for SBP and DBP estimation. The machine learning model can be trained for SBP and DBP estimation using a blood pressure (BP) training set that incorporates a PPG-signal set and a rPPG-signals set. Once the machine learning model has been trained, the blood pressure estimator 140 is configured to utilize the trained machine learning model to estimate SBP and DBP for the subject based on the rPPG signal and the derivative information from the rPPG-signal extraction system 130.

The report output module 150 communicates with the blood pressure estimator 140 for receiving an inference result with SBP and DBP estimation. The report output module 150 is configured to compile and present the result in a user-friendly format, providing a readable report regarding the subject's cardiovascular activity status.

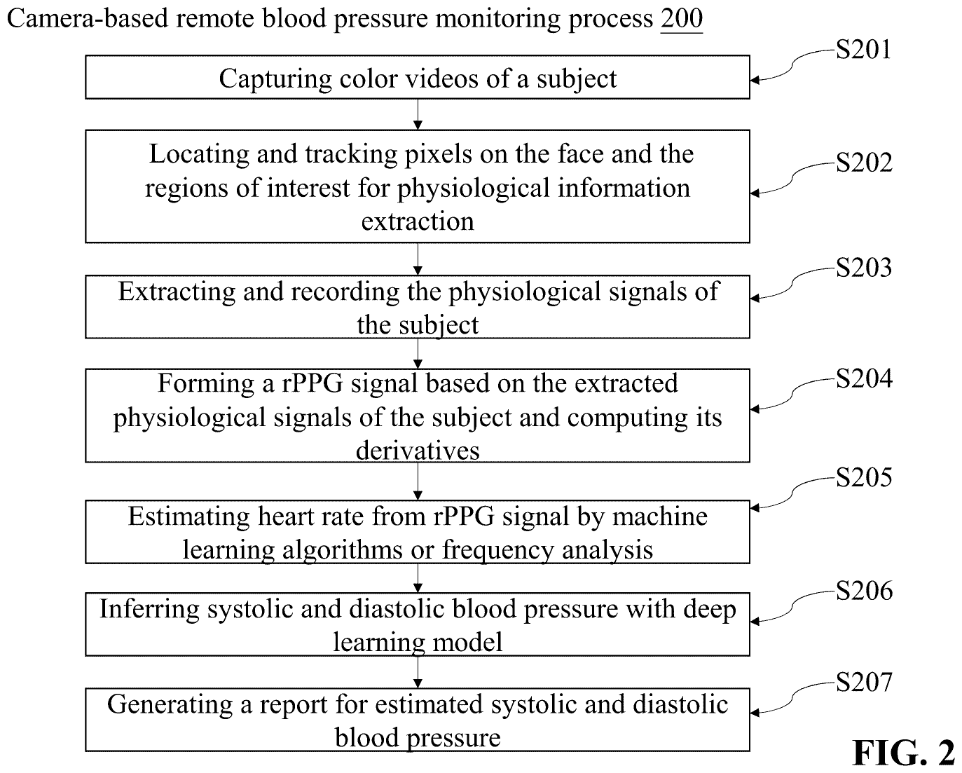

FIG. 2 shows a schematic diagram of a process 200 of estimating the SBP and DBP according to some embodiments of the present invention. The process 200 shown in FIG. 2 can be executed using the camera-based remote blood pressure monitoring system 100 as afore-mentioned in FIG. 1. The process 200 includes steps S201, S202, S203, S204, S205, S206, S207 for SBP and DBP estimation of a subject.

The process begins with step S201, which involves capturing color videos of a subject. During this step, the camera 110 is activated to record color videos for the subject, obtaining raw color frames that are essential for subsequent stages of physiological signal extraction.

In step S202, the raw color frames captured by the camera 110 are transmitted to the color image and signal processing system 120 for feature extraction and region identification. The color image and signal processing system 120 locates and tracks pixels on the subject's face and identifies regions of interest (ROIs) within the raw color frames for physiological information extraction.

Once the ROI of the subject's face is selected/determined, the process moves to step S203. The color image and signal processing system 120 extracts physiological signals from the ROI based on the color image frames and records these signals. During this stage, transformation of raw color frames into physiological signals, executed by the color image and signal processing system 120, helps minimize noise in the frames and provides higher-quality data for physiological signal extraction.

Next, the process advances to step S204. In step S204, the extracted physiological signals are transmitted from the color image and signal processing system 120 to the rPPG-signal extraction system 130. The rPPG-signal extraction system 130 processes the physiological signals to form a rPPG signal, which is a single 1D input with embedded physiological information of the subject. In this stage, the rPPG signal's derivatives of different orders are also computed by the rPPG-signal extraction system 130, acting as inputs to be fed into the machine learning model of the blood pressure estimator 140.

After formation/generation of the rPPG signal, the process proceeds to step S205. In step S205, heart rate (HR) of the subject is estimated from the rPPG signal by using machine learning algorithms or frequency analysis executed by the rPPG-signal extraction system 130, servings as inputs to be fed into the machine learning model of the blood pressure estimator 140.

Thereafter, the process advances to step S206. The rPPG signal, along with its derivatives of different orders, and an estimated HR from the corresponding rPPG signal are all fed into the machine learning model of the blood pressure estimator 140. The blood pressure estimator 140 utilizes the input data to perform SBP and DBP estimation. This stage employs machine learning and/or deep learning techniques, and the blood pressure estimator 140 applies a pre-trained model optimized for SBP and DBP estimation.

Finally, in step S207, the SBP and DBP estimation made by the blood pressure estimator 140 is output to the report output module 150. The report output module 150 generates and delivers a report, completing the process of estimating the subject's SBP and DBP.

The following will describe the details of how each component processes the data.

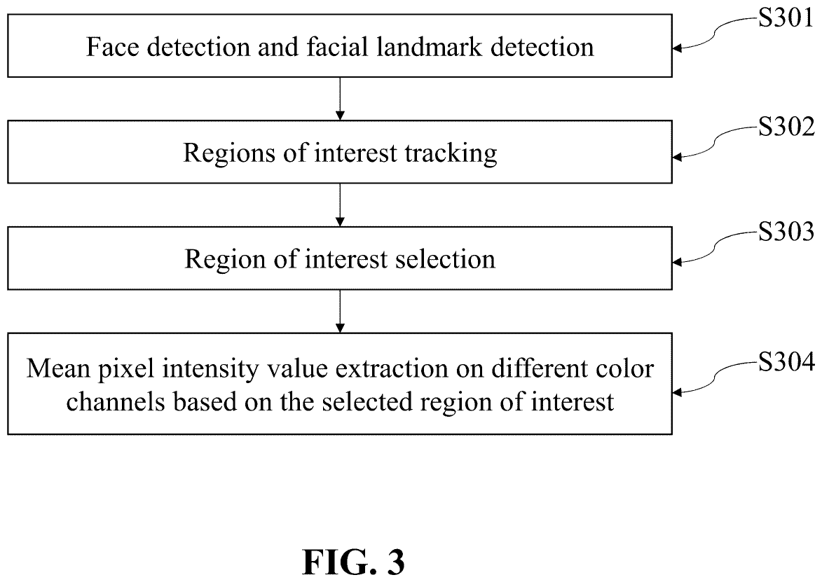

FIG. 3 shows a schematic diagram of a process for converting raw color frames into physiological signals using a color image and signal processing system 120 according to some embodiments of the present invention. The process includes steps S301, S302, S303, S304, S305.

Once the color image and signal processing system 120 receives the raw color frames of the subject from the camera 110, the process begins with step S301. In step S301, the color image and signal processing system 120 applies face detection and facial landmark detection to the raw color frames. In one embodiment, face detection and facial landmark detection are performed using computer vision techniques to identify the subject's face and facial landmarks in the color frames. The detection process utilizes various face detection and landmark identification methods to determine the location of the subject's facial features. For example, the color image and signal processing system 120 includes a feature extraction and pattern recognition-based model or a face detection model (e.g., a pre-trained convolutional neural network-based face detection model) for face detection and facial landmark detection.

Upon detecting the subject's face and facial landmarks, regions of interest (ROIs) are identified for face representation. In this regard, the designated ROIs serve as the basis for extracting physiological signals in subsequent processing.

Within the face representation, ROIs are determined using computer vision algorithms that approximate facial landmarks, which act as descriptors for the positions of key facial features such as the nose, eyes, and mouth.

For example, in the face detection and facial landmarks detection process, the raw color frames are first processed using the face detection model, which detects the bounding box of the face representation within the raw color frames. Once the face representation is localized, a facial landmark predictor applies a detection algorithm to identify key facial points, such as areas around the eyes, the tip of the nose, and regions near the mouth, and to determine facial landmarks, defining ROIs on the face representation for subsequent analysis.

The process moves to step S302. ROI tracking is performed. The identified ROIs on the face representation are continuously tracked throughout the captured video (i.e., the continuous color frames) to minimize noise and enhance the quality of the extracted physiological signals to be generated. The tracking process is based on the approximated facial landmarks.

In one embodiment, tracking the ROIs based on the detected facial landmarks includes tracking any ROI on the face representation using any kind of ROI tracking technique. For example, optical flow methods can be used to estimate the motion of facial features between consecutive frames (i.e., one frame and the next one frame). Alternatively, a filter can be applied to predict and correct the position of ROIs over time, enhancing robustness against minor occlusions or sudden movements.

The process moves to step S303. ROI selection is performed to identify the optimal region of interest for physiological signal extraction. The selection process is based on predefined metrics, such as signal-to-noise ratio (SNR), to minimize noise and enhance signal quality. Various selection methods can be employed depending on the actual requirements. For example, a max-SNR approach can be used, where multiple candidate ROIs are evaluated, and the one with the highest SNR is selected. In some embodiments, a machine learning-based method, such as a classification model trained on labelled data, can predict the high reliable ROI based on historical signal quality.

The process moves to the final step S304. A pixel intensity extraction process is performed, involving computing mean intensity values for each color channel (i.e., different color channels) within the selected region of interest. The mean intensity values are recorded and analyzed as they serve as core features for extracting physiological signals. Specifically, the mean intensity values can be treated as physiological signals, as the mean intensity values capture color variations caused by blood volume changes. The mean intensity values are then used in subsequent processing to generate rPPG signal.

In order to generate rPPG signals, extracting the mean pixel intensity values from the green channel is given high priority due to its greater sensitivity to blood volume changes under typical illumination conditions. However, in low-light environments, the red or blue channels may offer better signal fidelity. In some embodiments, the mean pixel intensity values from all three-color channels (red, green, and blue) can be obtained simultaneously, forming a three-channel signal representation for physiological signals.

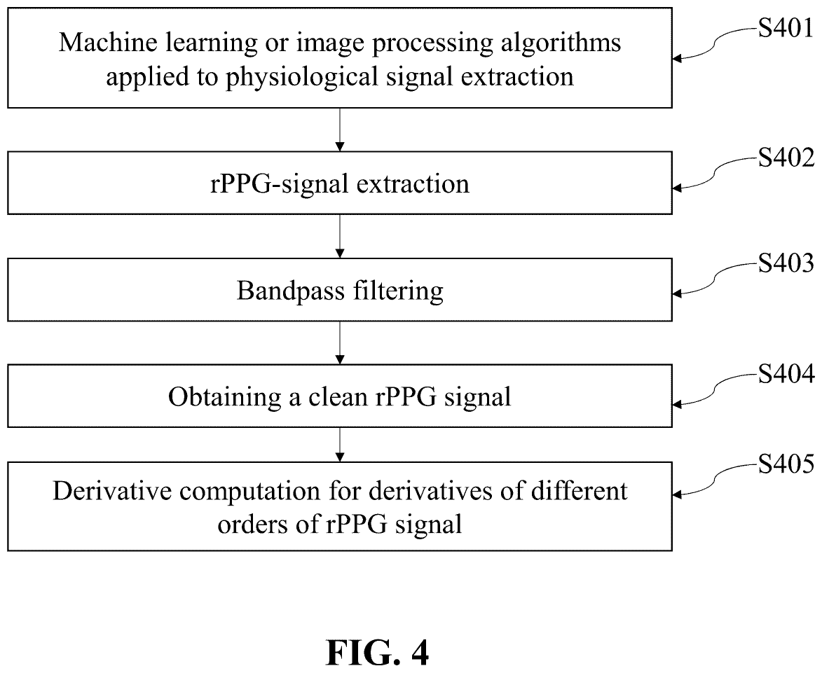

FIG. 4 shows a schematic diagram of a process for generating a rPPG signal from extracted physiological signals using a rPPG-signal extraction system 130 of the subject according to some embodiments of the present invention. The extraction includes steps S401, S402, S403, S404, S405 for generating a rPPG signal.

Once the rPPG-signal extraction system 130 receives the extracted physiological signals from the color image and signal processing system 120, the process begins with step S401. Based on the physiological signals, the rPPG-signal extraction system 130 takes the mean pixel intensity values of the different color channels of the selected region of interest to form 1D signals. In this regard, a 1D signal is generated by processing the mean pixel intensity values of a single-color channel over time. For each video frame, the mean pixel intensity value is calculated within the selected ROI. All mean intensity values, recorded frame by frame, form a continuous time series for each color channel. As a result, three independent 1D signals are produced, corresponding to the red, green, and blue channels, encoding physiological information collectively. The ID signals for all color channels are fed into machine learning or image processing algorithms.

The process moves to step S402. Machine learning or image processing algorithms are applied for processing the 1D signals to extract a rPPG signal. For example, a machine learning algorithm in this step may include a feature extraction model utilizing principal component analysis (PCA). The feature extraction model can process the 1D signals obtained from different color channels and extract the most significant components representing physiological variations. By identifying the dominant signal patterns, the feature extraction model isolates the periodic variations associated with blood volume changes. The resulting optimized signal components are then combined to generate a rPPG signal, which can be further analyzed for heart rate estimation and other physiological metrics.

The process moves to step S403. The rPPG-signal extraction system 130 has a bandpass filter, and the extracted rPPG signal is processed by the bandpass filter to remove noise and retain physiological-related information. In some embodiments, the bandpass filtering performed by the rPPG signal extraction system 130 utilizes a bandpass filter with any order, low cutoff frequency and high cutoff frequency to remove noise and retain physiological-related information.

The process moves to step S404. Upon the bandpass filtering, a clean rPPG signal is obtained, meaning that unwanted noise components, such as motion artifacts and environmental lighting fluctuations, are removed while preserving the physiological-related frequency range corresponding to heart rate variations.

The process moves to step S405. Derivative computation for different orders of the clean rPPG signal is performed. The computation of these derivatives helps capture the rate of change and dynamic variations in the clean rPPG signal, providing features for physiological analysis. For example, the first-order derivative highlights rapid fluctuations related to heartbeats, while the second-order derivative emphasizes acceleration and deceleration patterns in blood volume changes.

In some embodiments, the order of derivative computation is not limited to the first and second orders, as it may include derivatives of any order. In this regard, any order may encompass the first-order derivative (velocity of signal change), the second-order derivative (acceleration of signal change), or even higher-order derivatives for more advanced signal characterization.

Moreover, after computing the derivatives, estimating HR from the clean rPPG signal is performed using machine learning algorithms or frequency analysis. In some embodiments, frequency analysis methods, such as fast Fourier transform (FFT) or wavelet transform, identify the dominant frequency corresponding to heartbeats, filtering out non-cardiac noise. In some embodiments, machine learning models can estimate HR by learning signal patterns while compensating for motion artifacts and illumination changes.

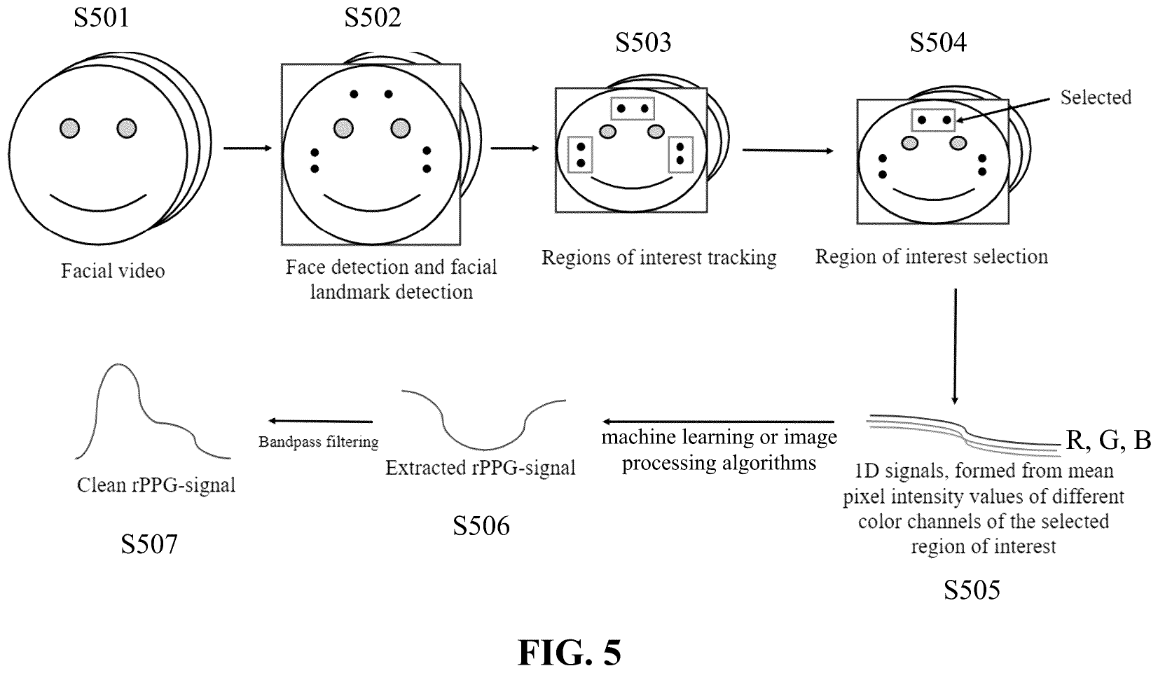

FIG. 5 illustrates a process of forming a rPPG signal from a captured video of a subject according to some embodiments of the present invention. The specific intermediate and final products of steps as described in FIG. 3 and FIG. 4 are visually shown in FIG. 5.

Step S501 and Step S502: The process of forming a clean rPPG signal begins with applying face detection and facial landmark detection on the captured video of the subject. Step S503: The regions of interest are then tracked continuously along the captured video based on the detected facial landmarks. Step S504: Certain metrics such as signal-to-noise ratio are used to select the best region of interest with the least noise. Step S505: 1D signals are formed based on the extracted mean of pixel intensity values of different color channels of the selected region of interest. For example, there are three color channels with red, green, and blue. Step S506: All these 1D signals are processed together with machine learning or image processing algorithms to extract the rPPG signal. Step S507: the clean rPPG signal is obtained by applying bandpass filtering on the extracted rPPG signal.

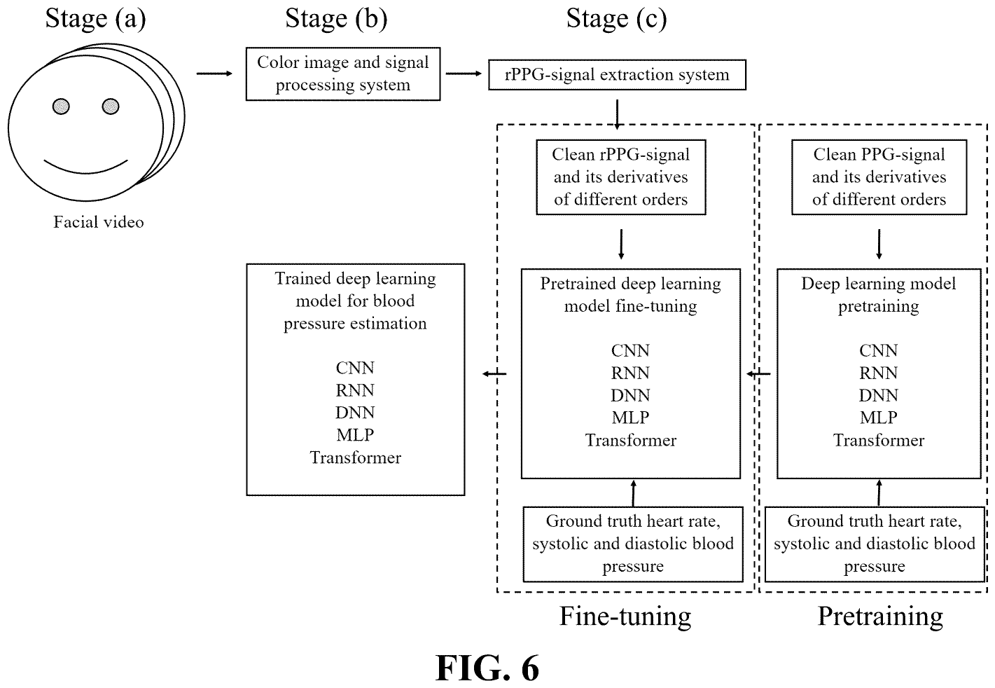

FIG. 6 illustrates a process of training a machine learning model of a blood pressure estimator using a captured video of a subject according to some embodiments of the present invention. Before training a machine learning model using rPPG signals and other ground truth parameters, stages (a), (b), (c) are executed.

The process of training a machine learning model for SBP and DBP estimation begins with stage (a) which is obtaining a facial video using the camera 110. Then, in stage (b), the color image and signal processing system 120 is applied to extract physiological signals from facial videos. In stage (c), the extracted physiological signals are passed to the rPPG-signal extraction system 130 to form clean rPPG signals and their derivatives of different orders.

Before training, a training set containing ground truth HR, SBP, and DBP values along with processed PPG and rPPG signals and their derivatives, corresponding to the same group of subjects, is well-prepared. In some embodiments, the machine learning model integrates convolutional neural networks (CNN), recurrent neural networks (RNN), deep neural networks (DNN), multi-layer perceptrons (MLP), transformer models, or combinations thereof.

During the training process, the machine learning model is first pretrained with clean PPG signals, the derivatives of different orders of the PPG signals, HR, SBP, and DBP ground truth values to learn the general features. To prepare the training data, clean PPG signals are first collected using contact-based sensors and processed with bandpass filtering to remove noise. The first-order and second-order derivatives of the PPG signals are then computed to capture rate changes and acceleration patterns. HR ground truth values are obtained from physical measurement devices and synchronized with the PPG signals. SBP and DBP ground truth values are recorded using physical monitoring devices and matched with the corresponding PPG segments. As such, the machine learning model is pretrained with properly aligned physiological signals.

The pretrained machine learning model is further fine-tuned with clean rPPG signals, the derivatives of different orders of the rPPG signals, HR, SBP, and DBP ground truth values. The fine-tuning process uses clean rPPG signals and their derivatives, which can be obtained through the same steps as afore-described. Also, the HR, SBP, and DBP ground truth values are collected through actual physiological measurements, similar to the pretrained stage, keeping alignment with real physiological data for the clean rPPG signals and their derivatives.

The machine learning can automatically extract features from the rPPG signals, their derivatives of different orders and HR, to learn to form a function to map these inputs to an estimated SBP and DBP. In some embodiments, the machine learning model can utilize the provided HR, SBP and DBP ground truth values to guide itself to improve the proposed function. Once the machine learning model is trained, it can be used to estimate SBP and DBP for a subject.

Once the model training is completed, the system can estimate blood pressure for a subject by simply recording a facial video. The recorded video is processed through the color image and signal processing system, followed by the rPPG-signal extraction system, which extracts physiological signals. These signals are then fed into the trained machine learning model, which analyzes the data and outputs a report containing SBP and DBP values.

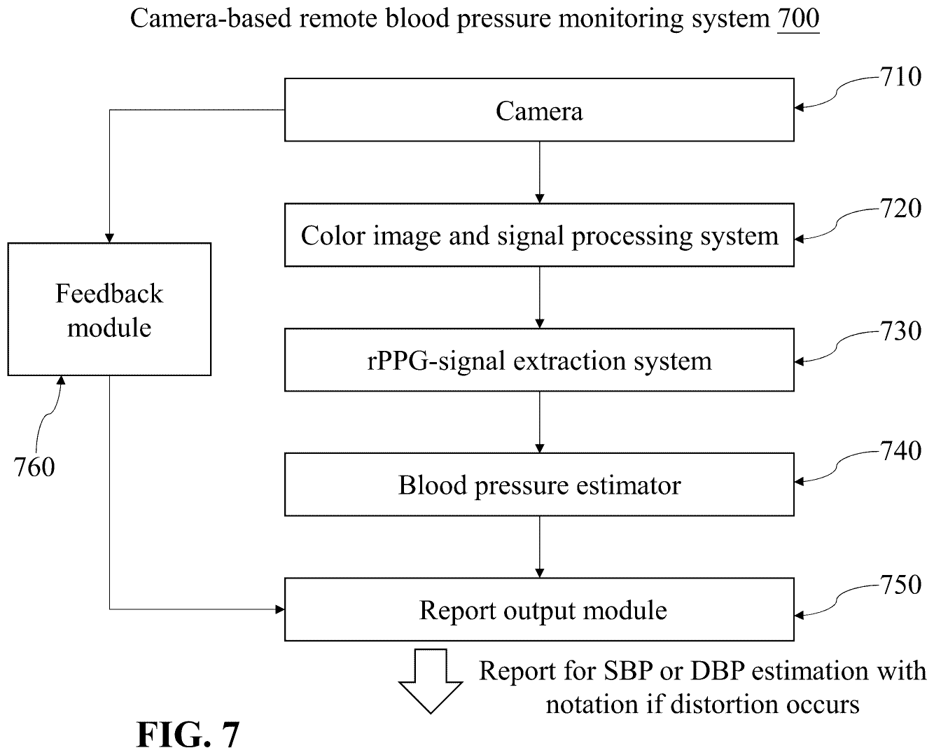

FIG. 7 illustrates a schematic diagram of an architecture of a camera-based remote blood pressure monitoring system 700 according to some embodiments of the present invention. The system 700 includes a camera 710, a color image and signal processing system 720, an rPPG-signal extraction system 730, a blood pressure estimator 740, and a report output module 750. These components can perform various steps and execute instructions as afore-described, including capturing a subject's facial video using the camera 710.

Similarly, the captured video is then processed by the color image and signal processing system 720 to extract relevant physiological features. The rPPG-signal extraction system 730 further processes these features to generate clean rPPG signals and their derivatives. These processed signals are then fed into the blood pressure estimator 740, where machine learning algorithms or signal analysis techniques estimate SBP and DBP. Finally, the report output module 750 generates a blood pressure measurement report.

Moreover, the system 700 further includes a feedback module 760, which analyzes the captured video from the camera 710 to assess the subject's physiological state. For example, the feedback module 760 can detect whether the subject has recently engaged in physical activity or whether the subject is experiencing conditions that may cause distortions in rPPG signal quality, potentially affecting the accuracy of SBP and DBP estimation.

In one embodiment, the feedback module 760 includes a post-exercise state detection model that analyzes multiple physiological and visual indicators to assess whether the subject has recently engaged in physical activity.

The post-exercise state detection model leverages computer vision and machine learning techniques to detect relevant changes in respiration rate, skin reflectance, facial expressions, and multimodal fusion-based classification to improve detection accuracy. Upon exercise, breathing patterns change, resulting in a faster and more pronounced respiration rate (RR). The feedback module 760 can utilize facial micro-movements analysis to estimate RR and detect irregularities. Increased perspiration after physical activity can make the skin appear shinier, particularly on the forehead and nose. The feedback module 760 applies specular reflection analysis and machine learning models to detect variations in skin brightness.

In one embodiment, the feedback module 760 is capable of detecting foreign objects on the face, such as glasses, masks, or facial coverings, which may interfere with accurate rPPG signal extraction. By utilizing computer vision algorithms and machine learning models, the feedback module 760 can identify and assess obstructions that could impact signal quality.

In one embodiment, the feedback module 760 can detect improper posture during measurement, ensuring optimal conditions for rPPG signal extraction. By analyzing head position, tilt angle, and stability using pose estimation techniques and facial landmark tracking, the feedback module 760 can determine if the subject is misaligned, moving excessively, or not facing the camera properly.

The report output module 750 is further configured to improve the reliability of blood pressure reporting. When the estimated SBP or DBP values fall outside the normal physiological range or exhibit an unusually high rate of change, the report output module 750 interacts with the feedback module 760 to verify whether the subject is experiencing conditions that may cause distortions in rPPG signal quality. If the feedback module 760 detects potential sources of measurement distortion, such as recent physical activity, facial obstructions, improper posture, or excessive movement, the report output module 750 includes a notation in the report, indicating the potential distortion factors that occurred.

This approach enables the efficient utilization of video data from multiple perspectives, enhancing the reliability of non-contact blood pressure measurement. By integrating the feedback module 760 and report output module 750, the system 700 can not only extract rPPG signals for blood pressure estimation but also validate measurement conditions and detect potential distortions.

The functional units and modules of the apparatuses and methods in accordance with the embodiments disclosed herein may be implemented using computing devices, computer processors, or electronic circuitries including but not limited to application specific integrated circuits (ASIC), field programmable gate arrays (FPGA), microcontrollers, and other programmable logic devices configured or programmed according to the teachings of the present disclosure. Computer instructions or software codes executing in the computing devices, computer processors, or programmable logic devices can readily be prepared by practitioners skilled in the software or electronic art based on the teachings of the present disclosure.

All or portions of the methods in accordance with the embodiments may be executed in one or more computing devices including server computers, personal computers, laptop computers, mobile computing devices such as smartphones and tablet computers.

The embodiments may include computer storage media, transient and non-transient memory devices having computer instructions or software codes stored therein, which can be used to program or configure the computing devices, computer processors, or electronic circuitries to perform any of the processes of the present invention. The storage media, transient and non-transient memory devices can be included, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

Each of the functional units and modules in accordance with various embodiments also may be implemented in distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

What is claimed is:

1. A system for camera-based remote blood pressure monitoring, comprising:
a camera configured to capture color image frames of a subject;
a color image and signal processing system configured to extract physiological signals according to the color image frames of the subject by:
executing light intensity analysis for the color image frames of the subject;
identifying a location of face and facial landmarks of the subject;
tracking regions of interest (ROIs) based on the approximated facial landmarks;
selecting one of the regions of interest; and
extracting the physiological signals from the selected region of interest;
a remote photoplethysmography-signal (rPPG-signal) extraction system configured to use the extracted physiological signals to generate a rPPG signal embedding the subject's cardiovascular activity information by:
forming 1D (one-dimension) signals from the extracted physiological signals;
applying machine learning or image processing algorithms to obtain a rPPG signal using the 1D signals;
applying bandpass filtering on the extracted rPPG signal; and
computing derivatives of different orders of the rPPG signal to generate a derivative information;
a blood pressure estimator receiving the rPPG signal and the derivative information and configured to output an estimated systolic blood pressure (SBP) and diastolic blood pressure (DBP) of the subject based on the rPPG signal and the derivative information using a well-trained machine learning model; and
a report output module configured to receive the estimated SBP and DBP of the subject and provide a readable report regarding the subject's SBP and DBP estimation values.

2. The system according to claim 1, wherein the color image and signal processing system comprises a feature extraction and pattern recognition-based model or a face detection model for face detection and facial landmark detection.

3. The system according to claim 2, wherein the regions of interest are identified by the color image and signal processing system, and the color image and signal processing system further comprises a facial landmark predictor applying a detection algorithm to identify key facial points, including areas around eyes, tip of nose, and regions near mouth of the subject.

4. The system according to claim 3, wherein the color image and signal processing system continuously tracks the identified regions of interest throughout the captured continuous color image frames.

5. The system according to claim 1, wherein the color image and signal processing system selects the region of interest based on a max-SNR metric by evaluating multiple candidate ROIs and selecting the one with the highest SNR.

6. The system according to claim 1, wherein the bandpass filtering is applied to the rPPG signal to obtain a clean rPPG signal by removing unwanted noise components while preserving a physiological-related frequency range.

7. The system according to claim 1, further comprising:
a feedback module configured to analyze a video captured by the camera to assess a physiological state of the subject, wherein the feedback module detects whether the subject has recently engaged in physical activity or whether the subject is experiencing conditions that causes distortions in quality of the rPPG signal.

8. The system according to claim 7, wherein, when the SBP and DBP estimation values fall outside a normal physiological range or exhibit an unusually high rate of change, the report output module is configured to interact with the feedback module to verify whether the subject is experiencing conditions that causes distortions in rPPG signal quality.

9. The system according to claim 8, wherein, if the feedback module detects potential sources of measurement distortion, the report output module is further configured to add a notation in the report, indicating potential distortion factors that occurred.

10. The system according to claim 1, wherein the well-trained machine learning model of the blood pressure estimator is trained for SBP and DBP estimation using a blood pressure (BP) training set containing a PPG-signal set, a rPPG-signals set, a set of derivatives of different orders, and ground truth values for heart rate, SBP, and DBP, all corresponding to the same group of subjects.

11. A method for camera-based remote blood pressure monitoring, comprising:
capturing color image frames of a subject using a camera;
extracting physiological signals according to the color image frames of the subject using a color image and signal processing system by steps of:
executing light intensity analysis for the color image frames of the subject;
identifying a location of face and facial landmarks of the subject;
tracking regions of interest (ROIs) based on the approximated facial landmarks;
selecting one of the regions of interest; and
extracting the physiological signals from the selected region of interest;
using the extracted physiological signals to generate a remote photoplethysmography-signal (rPPG signal) embedding the subject's cardiovascular activity information, using a physiological activity image processing system, by steps of:
forming 1D (one-dimension) signals from the extracted physiological signals;
applying machine learning or image processing algorithms to obtain a rPPG signal using the 1D signals;
applying bandpass filtering on the extracted rPPG signal; and
computing derivatives of different orders of the rPPG signal to generate a derivative information;
receiving the rPPG signal and the derivative information by a blood pressure estimator;
outputting, by the blood pressure estimator, an estimated systolic blood pressure (SBP) and diastolic blood pressure (DBP) of the subject based on the rPPG signal and the derivative information using a well-trained machine learning model; and
receiving, by a report output module, the estimated SBP and DBP of the subject and provide a readable report regarding the subject's SBP and DBP estimation values.

12. The method according to claim 11, wherein the color image and signal processing system comprises a feature extraction and pattern recognition-based model or a face detection model for face detection and facial landmark detection.

13. The method according to claim 12, wherein the regions of interest are identified by the color image and signal processing system, and the color image and signal processing system further comprises a facial landmark predictor applying a detection algorithm to identify key facial points, including areas around eyes, tip of nose, and regions near mouth of the subject.

14. The method according to claim 13, further comprising:
continuously tracking, by the color image and signal processing system, the identified regions of interest throughout the captured continuous color image frames.

15. The method according to claim 11, wherein the color image and signal processing system selects the region of interest based on a max-SNR metric by evaluating multiple candidate ROIs and selecting the one with the highest SNR.

16. The method according to claim 11, wherein the bandpass filtering is applied to the rPPG signal to obtain a clean rPPG signal by removing unwanted noise components while preserving a physiological-related frequency range.

17. The method according to claim 11, further comprising:
analyzing, by a feedback module, a video captured by the camera to assess a physiological state of the subject, wherein the feedback module detects whether the subject has recently engaged in physical activity or whether the subject is experiencing conditions that causes distortions in quality of the rPPG signal.

18. The method according to claim 17, further comprising:
interacting, by the report output module, with the feedback module to verify whether the subject is experiencing conditions that cause distortions in rPPG signal quality, when the estimated SBP or DBP values fall outside a normal physiological range or exhibit an unusually high rate of change.

19. The method according to claim 8, further comprising:
adding a notation in the report by the report output module, if the feedback module detects potential sources of measurement distortion, indicating potential distortion factors that occurred.

20. The method according to claim 11, wherein the well-trained machine learning model of the blood pressure estimator is trained for SBP and DBP estimation using a blood pressure (BP) training set containing a PPG-signal set, a rPPG-signals set, a set of derivatives of different orders, and ground truth values for heart rate, SBP, and DBP, all corresponding to the same group of subjects.
